SEQUENCE LISTING (1 sequence)

SEQ ID NO: 1 - protein, 5 aa, Artificial Sequence
  artificial sequence where X is adamanane modified glycine
  misc_feature (5)..(5): Xaa can be any naturally occurring amino acid
DGGLX

(19) United States
(12) Patent Application Publication
(10) Pub. No.: US 2019/0307833 A1
(43) Pub. Date: Oct. 10, 2019

(54) TREATMENT OF AGE-RELATED MACULAR DEGENERATION WITH NEUROTROPHIC PEPTIDERGIC COMPOUND

(71) Applicant: The Research Foundation for Mental Hygiene, Inc., Menands, NY (US)

(72) Inventors: Khalid Iqbal, Staten Island, NY (US); Inge Grundke-Iqbal, Staten Island, NY (US)

(73) Assignee: The Research Foundation for Mental Hygiene, Inc., Menands, NY (US)

(21) Appl. No.: 16/373,994

(22) Filed: Apr. 3, 2019

Related U.S. Application Data
(60) Provisional application No. 62/653,112, filed on Apr. 5, 2018.

(51) Int. Cl. A61K 38/08; A61P 27/02; A61K 9/00

(52) U.S. Cl. CPC A61K 38/08; A61P 27/02; A61K 9/0019; A61K 9/0048; A61K 9/0053

(57) ABSTRACT

The treatment of age-related macular degeneration with the chronic administration of the neurotrophic peptidergic compound P021. Chronic treatment of animal models for age-related macular degeneration prevented the pathological changes associated with age-related macular degeneration, including photoreceptor degeneration, lipofuscin granules, vacuoles and atrophy in retinal pigment epithelium (RPE), Bruch's membrane (BM) thickening, rosette-like structure formation, microgliosis and astrogliosis.

### CROSS-REFERENCE TO RELATED APPLICATIONS

The present application claims priority to U.S. Provisional App. No. 62/653,112, filed on Apr. 5, 2018.

### BACKGROUND OF THE INVENTION

### 1. Field of the Invention

The present invention relates to the treatment of age-related macular degeneration and, more particularly, to the use of a small peptidergic compound derived from ciliary neurotrophic factor to inhibit and treat age-related macular degeneration.

### 2. Description of the Related Art

Age-related macular degeneration (AMD)—also called macular degeneration—is the deterioration of the macula, which is the small central area of the retina that controls visual acuity. Macular degeneration is a leading cause of vision loss in Americans 60 years of age and older. AMD is an irreversible destruction of the macula, which leads to loss of the sharp, fine-detail, “straight ahead” vision required for activities such as driving, reading, recognizing faces, and seeing the world in color.

Age is a major risk factor for AMD. Its prevalence increases with aging, and according to some recent studies, its incidence is likely to increase significantly over the next 40 years. The number of people living with AMD is estimated to reach 196 million worldwide by 2020 and 288 million by 2040, if no effective treatment is developed. The risk of getting advanced AMD increases from 2% at 50-59 years of age to nearly 30% at 75 years of age.

AMD can progress to a “dry”, non-neovascular form leading to atrophy of the retinal pigment epithelium (RPE), choriocapillaris, and photoreceptors, or to a more rapid “wet,” neovascular form, which occurs when new blood vessels invade from the choroid and penetrate Bruch's membrane (BM), resulting in vascular leakage, hemorrhage, and scarring. Dry AMD is much more common than wet AMD, but choroidal neovascularization (CNV) in wet AMD accounts for much of the vision loss. Dry AMD may result from the aging and thinning of macular tissues, deposition of pigment in the macula, or a combination of these two processes.

AMD is a multifactorial disease, and its pathogenesis remains largely unknown, implying a complex interplay of genetic, environmental, metabolic, and functional factors. A growing body of evidence suggests that the immune system plays a key role in triggering neuroinflammation in the retina in AMD development. There are no early biomarkers to anticipate AMD, and no FDA-approved treatments are available for dry macular degeneration, although a few are in clinical trials, and nutritional intervention may help prevent its progression to the wet form.

The eye shares many neural and vascular similarities with the brain; it offers a direct window to cerebral pathology. The development of ocular biomarkers can have implications in the discovery of treatment for Alzheimer's disease (AD). Similarities between AMD and AD include pathophysiology, degeneration, and underlying genetic and other risk factors. Both AD and AMD involve degeneration of central nervous system (CNS) tissue, deposition of protein aggregates including beta-amyloid (Aβ) and hyperphosphorylated tau, and neuroinflammation. AMD has also been referred to as AD of the eye.

Mounting evidence indicates that patients with AD and mild cognitive impairment (MCI) exhibit a wide spectrum of ocular abnormalities. Among the many characteristics it shares with the brain, the retina contains neurons, astroglia, microglia, microvasculature with similar morphological and physiological properties, and a blood barrier. Axons of the optic nerve connect the retina and brain directly and facilitate vesicular transport of amyloid precursor protein (APP) synthesized in retinal ganglion cells (RGCs). Furthermore, retinal neurons and glia express proteins that have been implicated in the amyloid cascade.

Despite lacking a macula, the retina of an old mouse shows many AMD features and has been useful in studying risk factors for AMD, including environment, age, genetics, diet, smoke, and inflammation. Several animal models of AMD have been generated and have revealed many important aspects about the underlying pathology of the disease and are thus useful for establishing the efficacy of AMD treatments

P021 is a small peptidergic compound derived from ciliary neurotrophic factor (CNTF) that is orally bioavailable and is blood brain barrier (BBB)—permeable; it enhances dentate gyrus neurogenesis and neuronal plasticity by competitively inhibiting the leukemia inhibitory factor and by increasing the expression of brain-derived neurotrophic factor (BDNF). In previous studies, both P021 and its parent non-adamantylated peptide were found to rescue cognitive impairment, synaptic deficit, neuroinflammation, and tau and Aβ pathologies in rat and mouse models of AD. To date P021 has not been explored in connection with the treatment of eye diseases such as AMD. Accordingly, there is a need in the art for a compound that can be used to inhibit and treat AMD.

### BRIEF SUMMARY OF THE INVENTION

The present invention is a method of treating a subject for age related macular degeneration that comprises the step of administering a therapeutic amount of a peptidergic compound having the formula Ac-DGGL^{A}G-NH₂ (SEQ ID NO: 1). The therapeutic amount may be a dosage of between 0.3 mg to 40 mg per kilogram of body weight. The administering of the therapeutic amount of the compound may occur on a daily basis for at least 90 days. The compound may be administered orally, but can be also administered intraocularly, intravenously or subcutaneously, with the dosage for intraocular, intravenous, and subcutaneous, and administration being towards the low end of the dosage range.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1A through 1G are a series of schematics, images, and graphs demonstrating that AMD-like retinal pathology is prevented by P021 treatment in aged rats. FIG. 1A is the experimental design to study the effect of P021 treatment on retina in aged rats. Fisher-rats at age ˜19-21 months received P021 in saline (500 nmoles/kg body weight/day, by gavage) or vehicle only for 3 months, whereas ˜2- to 3-month-old rats received vehicle only for 3 months. FIG. 1B is a schematic diagram showing the sections of rat eye employed in the study. FIG. 1C are representative images of the central and peripheral retinas showing the morphology of retinal layers, H & E staining in 5-μm sagittal paraffin sections. Rosette-like rearrangements of photoreceptor cells that extend from the ONL and across the OPL toward INL (C. b), and photoreceptor inner segments (ISs) that project inward to form the core of the rosette (blue arrow) were found in ˜22-24-month-old vehicle rats. FIG. 1D-G are the quantification of rows and thickness of the ONL and INL in central and peripheral retina (3-5 sections per rat, n=3-7; exact “n” values are labeled in each bar). Data in FIG. 1 are shown as mean±SEM. *P<0.05, **P<0.01, ***P<0.001 by one-way ANOVA followed by Newman-Keuls Multiple Comparison Test. AMD, age-related macular degeneration; ONL, outer nuclear layer (photoreceptor cell bodies); OPL, outer plexiform layer; INL, inner nuclear layer; IPL, inner plexiform layer; GCL, ganglion cell layer; m, months; H&E, hematoxylin and eosin.

FIGS. 2A through 2C are a series of images, and graphs showing that chronic treatment with P021 prevents AMD-like pathology of RPE in aged rats. FIG. 2A is H & E staining of RPE in the retina. AM were short or out of order, and atrophy and vacuolization of RPE (A. c, yellow arrow) were found in ˜22- to 24-month-old/vehicle rats. FIG. 2B are representative images of auto-fluorescence at λₑₓ=543 (red channel). The number of deposits between RPE and Cho (B. b, blue arrow), and lipofuscin granules were markedly greater in ˜22- to 24-month-old/vehicle rats than ˜5- to 6-month-old/vehicle rats or ˜22- to 24-month-old/P021 rats (B. b, c). FIG. 2C is the quantification of deposits number (2-3 sections per rat, n=7). Data are shown as mean±SEM. *P<0.05, **P<0.01 by one-way ANOVA followed by Newman-Keuls Multiple Comparison Test. RPE, retinal pigment epithelium; AM, apical microvilli; BM, Bruch's membrane; Cho, choroid; OS, outer segment.

FIG. 3A through 3F are a series of schematics, images, and graphs demonstrating that AMD-like retinal pathology is prevented by P021 treatment in aged 3×Tg-AD mice. FIG. 3A is an experimental design to study the effect of chronic treatment with P021 on retina in aged 3×Tg-AD mice. Starting at 3 months of age, WT or 3×Tg mice received mouse chow (AIN-76, Research Diets, New Brunswick, N.J.) without (vehicle) or containing P021 (60 nmoles/g diet). At 21 months of age, they were perfused with 0.1M PBS. A second group of WT and 3×Tg-AD mice at 3 months of age was employed as young controls. FIG. 3B is H & E staining to detect retinal morphology in 5-μm-thick sagittal paraffin sections; a-e. the sagittal eyecup sections parallel to the axis from pupil to optic nerve; b. Position of central and peripheral retinas are labeled in blue; f-j. Representative images of central retina; h, i. The extension of cell nuclei from the ONL to the INL (blue arrow); disruption and atrophy of RPE with increased brown lipofuscin granules (i. green “RPE”); k-o. Representative images of peripheral retina. FIG. 3C-F are the quantification of rows and thickness of the ONL and INL in the central and peripheral retina (3-5 sections per mouse, n=5-6; exact “n” values are labelled in each bar of panel FIG. 3C). Data are shown as mean±SEM. *P<0.05, **P<0.01 by one-way ANOVA followed by Newman-Keuls Multiple Comparison Test. AMD, age-related macular degeneration; WT, wild type; 3×Tg, triple-transgenic; AD, Alzheimer's disease; RPE, retinal pigment epithelium; OS, outer segment; IS, inner segment; ONL, outer nuclear layer; OPL, outer plexiform layer; INL, inner nuclear layer; IPL, inner plexiform layer; GCL, ganglion cell layer. NFL, nerve fiber layer.

FIGS. 4A through 4C are a series of images and graphs demonstrating that chronic treatment with P021 prevents AMD-like features of RPE in aged mice. FIG. 4A are representative images of RPE layer (A. d). Thickened (blue asterisk) and atrophic (blue “BM”) BM (A. e). 21-month-old 3×Tg/Veh mice showed disarrangement or thinned layer of RPE, full of many brown lipofuscin granules, more than in 21-month-old 3×Tg/P021 mice (A. g), whereas aged WT mice showed a little decrease in the thickness of RPE (A. c, blue arrow), as compared with young mice, which showed regular structure and normal arrangement of RPE with no or only a few lipofuscin granules (A. a, b). FIG. 4B is auto-fluorescence at λₑₓ=543 (red channel). Compared with 3-month-old mice and P021-treated 21-month-old 3×Tg-mice (B. a, b, f), more deposits (blue arrows) between RPE and Cho, and lipofuscin granules were detected in 21-month-old WT and 21-month-old 3×Tg-mice (B. c-e). FIG. 4C is the quantification of number of deposits in different groups of mice (2-3 sections per mouse, n=5). Data are shown as mean±SEM. *P<0.05, **P<0.01 by one-way ANOVA followed by Newman-Keuls Multiple Comparison Test. AMD, age-related macular degeneration; 3×Tg, triple-transgenic; RPE, retinal pigment epithelium; BM, Bruch's membrane; Cho, choroid.

FIG. 5A through 5D are a series of images and graphs demonstrating that P021 prevents microgliosis in the retinas of aged mice and rats. FIG. 5A are representative images of Iba-1 immunofluorescence in the central and peripheral retina of mice. Iba-1-positive staining (green) was confined mainly to the NFL and GCL in the retina of 3-month-old mice (A. a, b, f, g) and P021-treated 21-month-old mice (A. e, j), whereas it was widely distributed in all the retinal layers from the NFL to the ONL in 21-month-old WT/vehicle and 21-month-old 3×Tg/vehicle mice (A. c, d, h, i). FIG. 5B is the quantification of the area percentage of Iba-1 from NFL to ONL in the central (n=5-6) and peripheral (n=3-6) retinas of mice (2-3 sections per mouse). FIG. 5C are representative images of Iba-1 immunofluorescence in the central and peripheral retinas of rats. Iba-1-positive staining (green) was confined predominately from the NFL to the INL in the retinas of ˜5- to 6-month-old/Veh rats (C. a, d) and ˜22- to 24-month-old/P021 rats (C. c, f), whereas it was widely distributed in all the retinal layers from the NFL to the RPE, and in Cho in ˜22- to 24-month-old/Veh rats (C. b, e). FIG. 5D is the quantification of the area percentage of Iba-1 from the NFL to the OS in the central and peripheral retina of rats (2-3 sections per rat, n=6-7). The sections were counterstained with TO-PRO 3 iodide, a fluorescent nuclear stain. Data are shown as mean±SEM. *P<0.05, **P<0.01 and ***P<0.001 by one-way ANOVA followed by Newman-Keuls Multiple Comparison Test. WT, wild type; 3×Tg, triple-transgenic; AD, Alzheimer's disease; Cho, choroid; RPE, retinal pigment epithelium; OS, outer segment; IS, inner segment; ONL, outer nuclear layer; OPL, outer plexiform layer; INL, inner nuclear layer; IPL, inner plexiform layer; GCL, ganglion cell layer; NFL, nerve fiber layer.

FIG. 6A through 6D are a series of images and graphs demonstrating that P021 inhibits astrogliosis in the retinas of aged mice and rats. A, Representative images of GFAP immunofluorescence in the central and peripheral retina of mice. GFAP-positive staining (green) was confined predominately to the NFL and GCL in the retina of 3-month-old/vehicle WT and 3×Tg mice (A. a, b, f, g), whereas it was widely distributed from the NFL to the OPL in 21-month-old WT/Veh and 21-month-old 3×Tg/Veh mice (A. d, h, i) and was reduced in 21-month-old 3×Tg/P021 mice (A. e, j). FIG. 6B is the quantification of the area percentage of GFAP from the NFL to the ONL in the central (n=5-6) and peripheral (n=3-6) retinas of mice (2-3 sections per mouse). C. Representative images of GFAP immunofluorescence in the central and peripheral retinas of rats. GFAP-positive staining (green) was confined mainly from the NFL to the INL in the retinas of ˜5- to 6-month-old/Veh rats (C. a, d) and ˜22- to 24-month-old/P021 rats (C. c, f), whereas it was widely distributed from the NFL to the OPL in ˜22- to 24-month-old/Veh rats (C. b, e). FIG. 6D is the quantification of the area percentage of GFAP from the NFL to the OS in the central and peripheral retina of rats (2-3 sections per rat, n=6-7). The sections were counterstained with TO-PRO 3 iodide, a fluorescent nuclear stain. Data are shown as mean±SEM. *P<0.05, ** P<0.01 and ***P<0.001 followed by one-way ANOVA with Newman-Keuls Multiple Comparison Tests. WT, wild type; 3×Tg, triple-transgenic; GFAP, glial fibrillary acidic protein; Cho, choroid; RPE, retinal pigment epithelium; OS, outer segment; IS, inner segment; ONL, outer nuclear layer; OPL, outer plexiform layer; INL, inner nuclear layer; IPL, inner plexiform layer; GCL, ganglion cell layer, NFL, nerve fiber layer.

FIG. 7A thru 7E is a series of images and graphs showing that no primary antibody controls of immunofluorescence. A, B. Representative images of immunofluorescence of retinas of ˜22- to 24-month-old/Veh rats in the absence of any primary antibodies by using goat anti-rabbit (GAR) or goat anti-mouse (GAM) secondary antibodies. C, D. Representative images of immunofluorescence in the retinas of 3×Tg-21m/Veh mice by using GAR or GAM secondary antibody. E. Representative image of immunofluorescence in the optic nerves of 3×Tg-21m/Veh mice by using GAM secondary antibody. The sections were counterstained with TO-PRO 3 iodide, a fluorescent nuclear stain. GAR, Alexa Fluor 488-conjugated goat anti-rabbit IgG; GAM, Alexa Fluor 488-conjugated goat anti-mouse IgG; 3×Tg, triple-transgenic; IS, inner segment; ONL, outer nuclear layer; OPL, outer plexiform layer; INL, inner nuclear layer; IPL, inner plexiform layer; GCL, ganglion cell layer; NFL, nerve fiber layer.

FIG. 8A through 8B is series of images and graphs showing that P021 prevents the increase in total tau immunoreactivity in retinas of aged rats and mice. FIG. 8A are representative images of immunofluorescence (green) with rabbit polyclonal tau antibody R134d in the central (upper panels) and peripheral retinas (lower panels) of rats. FIG. 8B are representative images of R134d immunofluorescence in the central (upper panels) and peripheral retinas (lower panels) of mice. The sections were counterstained with TO-PRO 3 iodide, a fluorescent nuclear stain. WT, wild type; 3×Tg, triple-transgenic; m, month; Veh, vehicle; ONL, outer nuclear layer; OPL, outer plexiform layer; INL, inner nuclear layer; IPL, inner plexiform layer; GCL, ganglion cell layer; NFL, nerve fiber layer.

FIG. 9A through 9B is a series of graphs and images showing that P021 prevents increase in the expression of tau hyperphosphorylation at Ser-396/404 (PHF-1site) in retinas of aged rats and mice. FIG. 9A are representative images of PHF-1 immunofluorescence (green) in the central (upper panels) and peripheral retina (lower panels) of rats. FIG. 9B are representative images of PHF-1 immunofluorescence (green) in the central (upper panels) and optic nerve (lower panels) of mice. The sections were counterstained with TO-PRO 3 iodide, a fluorescent nuclear stain. WT, wild type; 3×Tg, triple-transgenic; m, month; Veh, vehicle; ONL, outer nuclear layer; OPL, outer plexiform layer; INL, inner nuclear layer; IPL, inner plexiform layer; GCL, ganglion cell layer; NFL, nerve fiber layer.

FIG. 10A through 10C is a series of images showing that P021 prevents increase in the expression of tau hyperphosphorylation at Ser-202/Thr-205 (AT8 site) in retinas of aged rats and mice. FIG. 10A are representative images of AT8 immunofluorescence in the central retinas of rats. FIG. 10B are representative images of AT8 immunofluorescence in the central and peripheral retinas of mice. FIG. 10C are representative images of AT8 immunofluorescence in the optic nerve of 3×Tg-mice. The sections were counterstained with TO-PRO 3 iodide, a fluorescent nuclear stain. WT, wild type; 3×Tg, triple-transgenic; m, month; Veh, vehicle; RPE, retinal pigment epithelium; OS, outer segment; IS, inner segment; ONL, outer nuclear layer; OPL, outer plexiform layer; INL, inner nuclear layer; IPL, inner plexiform layer; GCL, ganglion cell layer; NFL, nerve fiber layer.

FIGS. 11A through 11C is a series of images showing that P021 prevents increase of Aβ/APP immunoreactivity in retinas of aged rats and mice. FIG. 11A are representative images of immunofluorescence with anti Aβ (4G8) in the central retinas of rats. FIG. 11B are representative images of 4G8 immunofluorescence in the central retinas of mice. FIG. 11C are representative images of Aβ immunofluorescence in the central (upper panel) and peripheral (lower panel) retinas of mice. The sections were counterstained with TO-PRO 3 iodide, a fluorescent nuclear stain. m, month; Veh, vehicle; ONL, outer nuclear layer; OPL, outer plexiform layer; INL, inner nuclear layer; IPL, inner plexiform layer; GCL, ganglion cell layer; NFL, nerve fiber layer.

FIG. 12 is a series of images showing that P021 prevents increase in the expression of VEGF in retinas of aged mice. Representative images of VEGF immunofluorescence in central retinas (upper panel) and optic nerves of rats (lower panel). VEGF-positive particles were observed in different groups. The sections were counterstained with TO-PRO 3 iodide, a fluorescent nuclear stain. WT, wild type; 3×Tg, triple-transgenic; m, month; Veh, vehicle; RPE, retinal pigment epithelium; OS, outer segment; IS, inner segment; ONL, outer nuclear layer; OPL, outer plexiform layer; INL, inner nuclear layer; IPL, inner plexiform layer; GCL, ganglion cell layer; NFL, nerve fiber layer.

### DETAILED DESCRIPTION OF THE INVENTION

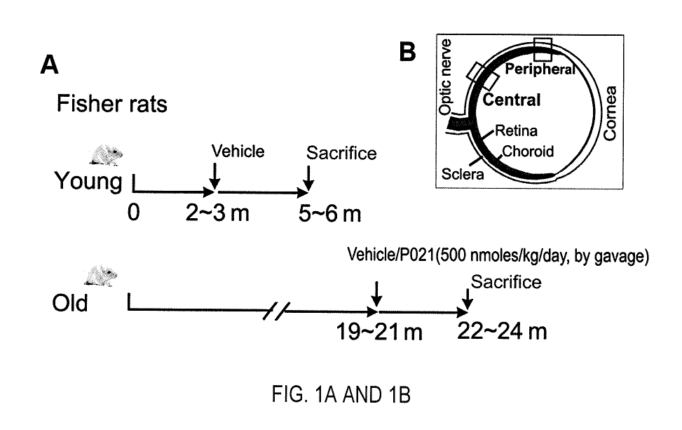

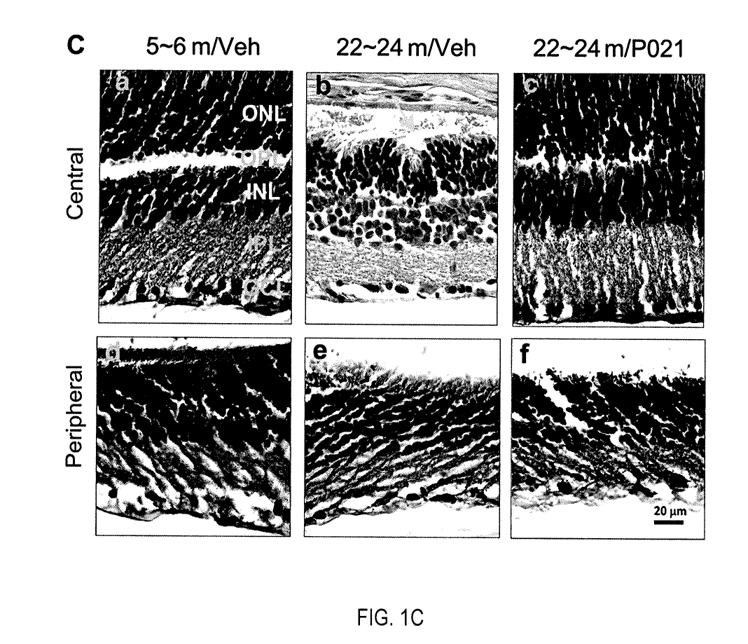

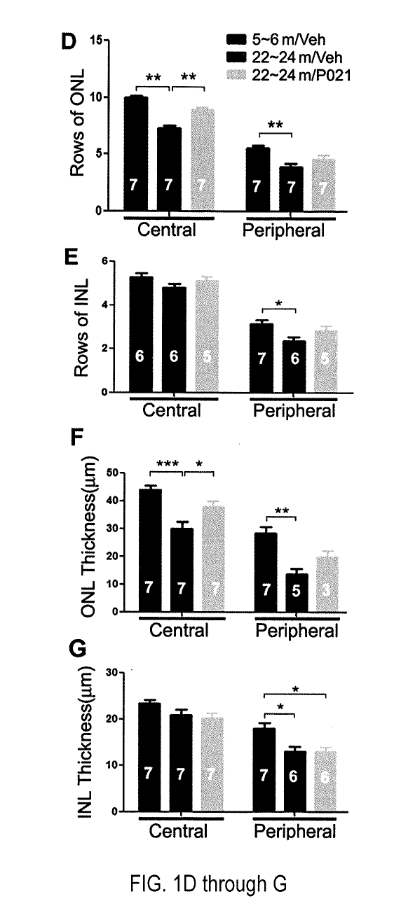

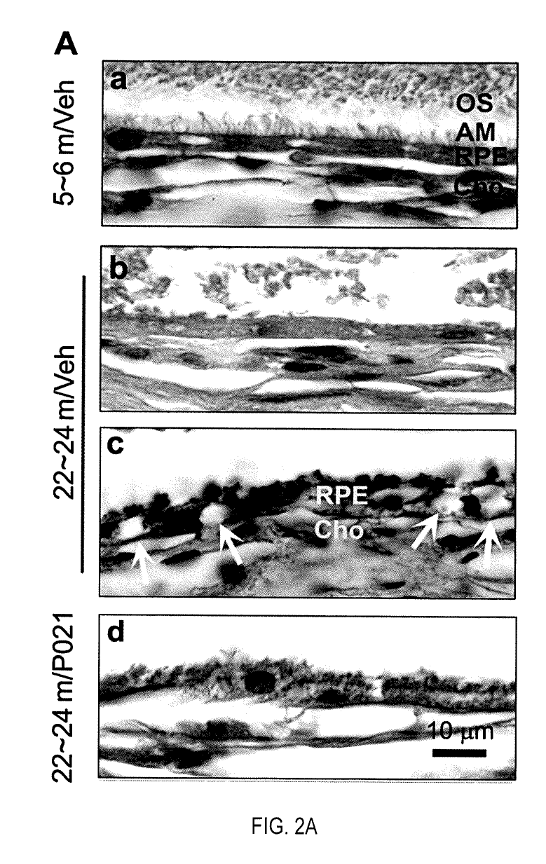

Referring to the figures, wherein like numeral refer to like parts throughout, there is seen in FIGS. 1 through 6 a series of experiments demonstrating that AMD-like retinal pathology was prevented by the chronic treatment of representative animal models with the neurotrophic peptidergic compound P021, which comprises the short peptide Ac-DGGL^{A}G-NH₂ (SEQ ID NO: 1) having an unnatural amino acid based upon adamantane located at the C-terminus, with the C-terminal amidated and the N-terminal acetylated. P021 may be synthesized by standard solid phase peptide synthesis (SPPS) methods following the Fmoc-strategy.

The experiments revealed photoreceptor degeneration, lipofuscin granules, vacuoles and atrophy in retinal pigment epithelium (RPE), Bruch's membrane (BM) thickening; and in aged rats rosette-like structure formation was found. Microgliosis and astrogliosis were observed in different retinal layers. In addition, total tau, phosphorylated tau, Aβ/APP, and VEGF were widely distributed in the sub-retina of aged rats and 3×Tg mice. Importantly, chronic treatment with P021 for 3 months in rats and 18 months in 3×Tg mice prevented the pathological changes above. The results of the experiments indicate a new therapeutic use of P021 for the prevention and treatment of AMD. For example, P021 may be used to treat a human for age related macular degeneration by administering a therapeutic amount of P021. A therapeutic amount may comprise a dose of 0.3 mg/kg to 40 mg/kg body weight, preferably 0.5 mg/kg to 10 mg/kg body weight. The administering of the therapeutic amount of the compound may occur on a daily basis for at least 90 days. The compound may be administered orally, but can be also administered intraocularly, intravenously or subcutaneously, with the dosage for intraocular, intravenous, and subcutaneous, and administration being towards the low end of the dosage range. The administration route may include standard delivery mechanism as well as liposomes or slow release formulations.

### Example

Material and Methods

Antibodies and Reagents

The primary antibodies used in this study are listed in Table 1.

TABLE 1
Primary antibodies used in this study

| Antibody | Specificity | Species | Type | Dilution |
|---|---|---|---|---|
| Iba-1 | Iba-1 | R | Poly- | 1:1000 |
| SMI22 | GFAP | M | Mono- | 1:2000 |
| R134d | Total tau | R | Poly- | 1:1000 |
| PHF-1 | P-tau (Ser396/404) | M | Mono- | 1:200 |
| AT-8 | P-tau (Ser202/T205) | M | Mono- | 1:1000 |
| 4G8 | Aβ/APP | M | Mono- | 1:1000 |
| Anti-Aβ | Aβ/APP | R | Mono- | 1:500 |
| Anti-VEGF(C-1) | VEGF | M | Mono- | 1:100 |

Key: Iba-1, ionized calcium binding adaptor molecule 1; GFAP, glial fibrillary acidic protein; VEGF, vascular endothelial growth factor; p-tau, phosphorylated tau; APP, amyloid precursor protein; Aβ, amyloid-β; Poly-, polyclonal; Mono-, monoclonal; R, rabbit; M, mouse.

Alexa Fluor 488-conjugated goat anti-mouse and Alexa Fluor 488-conjugated goat anti-rabbit IgG, TO-PRO™-3 Iodide, and ProLong Gold Anti-fade reagent were obtained from Thermo Fisher Scientific (Rockford, Ill., USA). Other chemicals were from Sigma-Aldrich (St. Louis, Mo., USA).

Synthesis and Features of P021

The peptidergic compound P021 (Ac-DGGLAG-NH2; mol. wt. of 578.3) corresponds to a biologically active region of human CNTF (amino acid residues 148-151) to which adamantylated glycine was added at the C-terminal to increase its stability and lipophilicity. The peptide was synthesized and purified by reversed-phase high-performance liquid chromatography to ˜96% purity, and the sequence of the peptide was confirmed by mass spectrometry, as described previously.

P021 is quite stable in artificial gastric juice (˜90% during 30 minutes) and in artificial intestinal juice (˜95% during 120 minutes). BBB studies on P021, which were carried out through a commercial service (APREDICA, Watertown, Mass., USA), demonstrated that a sufficient amount of P021 crossed the BBB to exert its effect in the brain.

Animals

Female Fisher Rats ˜19-21 months and ˜2-3 months of age (Charles River, France) weighing approximately 300 g were used.

Homozygous 3×Tg-AD mice harboring human APPSWE and tauP301L transgenes with knock-in PS1M146V under the control of the mouse Thy1.2 promoter, generated in the laboratory of Dr. Frank LaFerla, and age-matched control mice of the same genetic background (hybrid 129/Sv×C57BL/6) were obtained from the Jackson Laboratory (https://www.jax.org/strain/004807). Male and female 3×Tg-AD mice and age-matched control mice were bred in the animal colony of the New York State Institute for Basic Research in Developmental Disabilities (Staten Island, N.Y.).

Animals were housed at a standard temperature (22±1° C.) and in a light-controlled environment (lights on from 7 AM to 8 PM), had access to food and water ad libitum, and were housed (four or five animals per cage) in pathogen-free facilities with 12-hour light/12-hour dark cycles. All animal studies were carried out according to the National Institutes of Health guidelines for the care and use of laboratory animals and were approved by the National Animal Experiment Board, Finland.

Animal Treatment

Female aged (19˜21 months) Fisher rats (n=7) were given P021 per os by gavage (10 ml/kg body weight) once a day for 88 days. The dose of P021 was 500 nanomoles (289.15 microgram)/kg body weight daily. As controls, a second group of aged (19˜21 months) female rats (n=7) and a group of young adult female rats (2-3 months, n=7) were treated identically, but with normal saline (vehicle) only (FIG. 1A). Administration of vehicle and test compound was done at 7-9 AM daily in the pretesting phase.

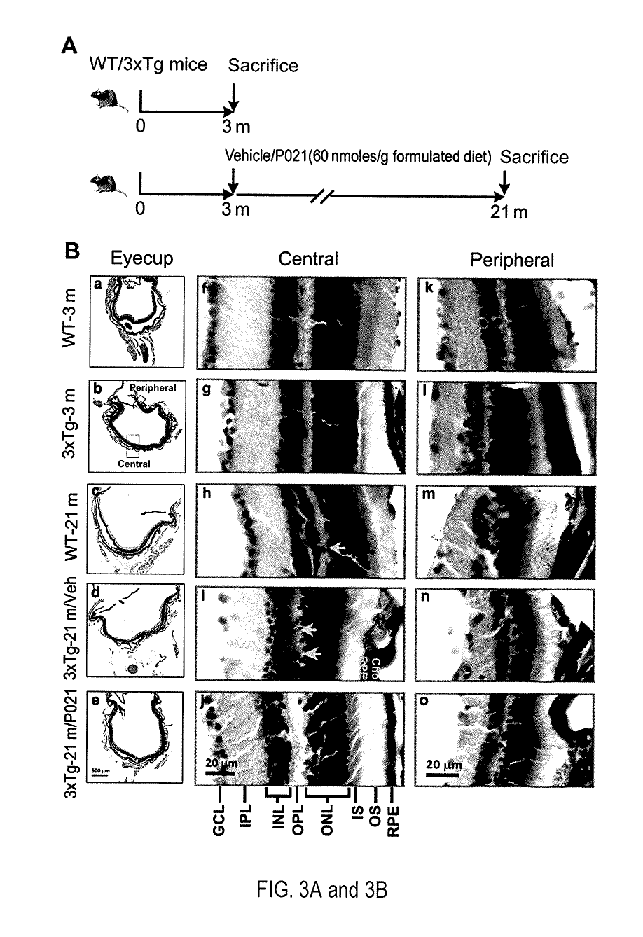

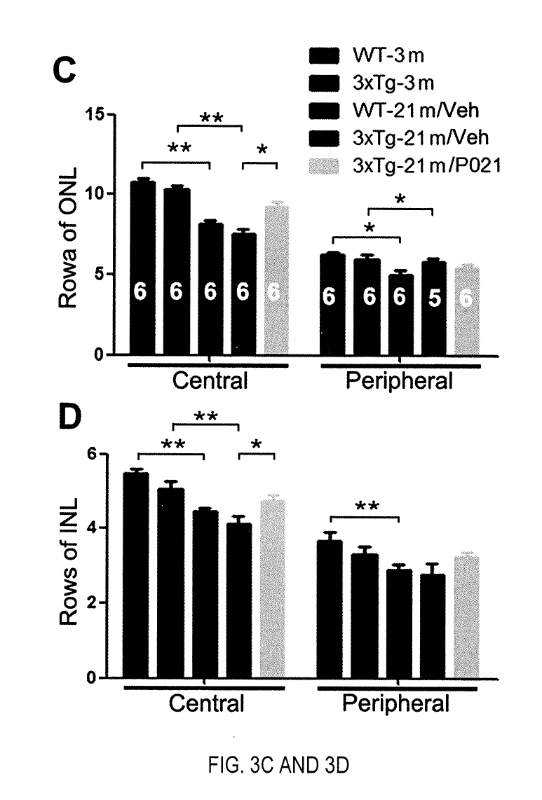

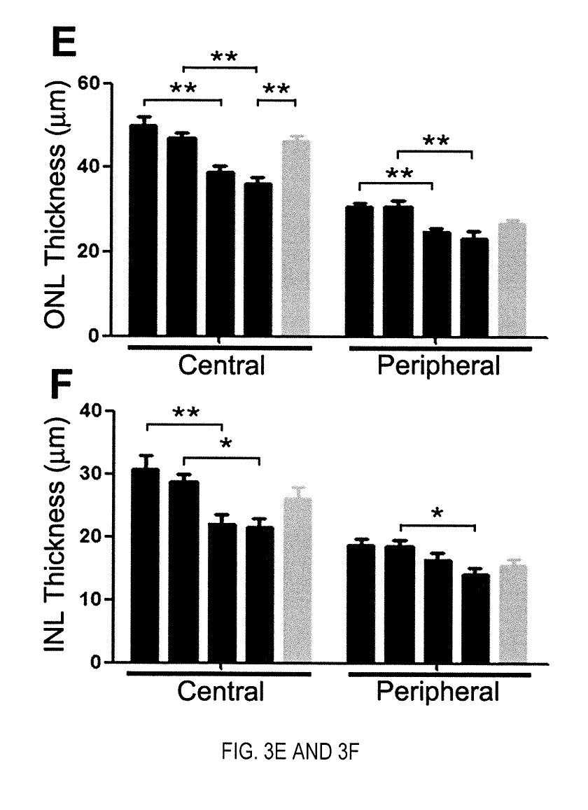

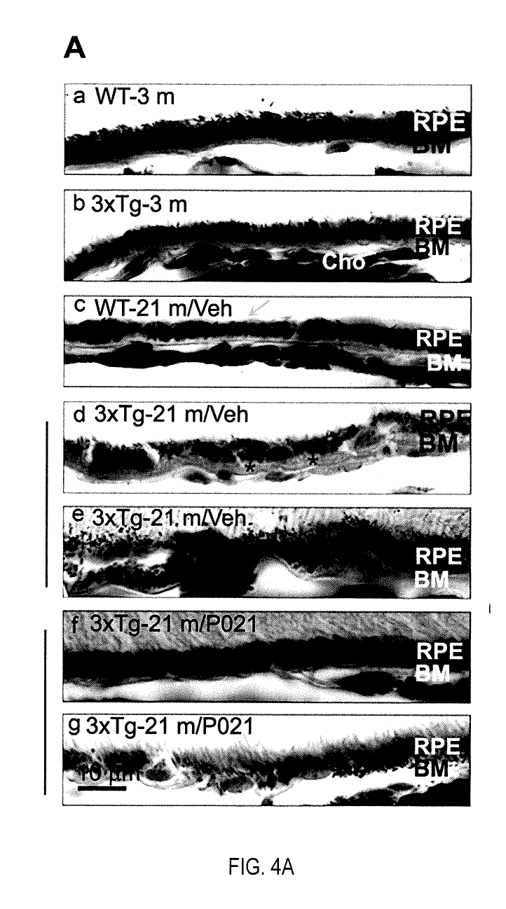

At approximately 3 months of age, the female wild type and 3×Tg-AD mice were divided into five groups (n=6-7 mice per group) (FIG. 3A): (1) Wild type mice, age 3 months, without treatment (WT-3m); (2) 3×Tg-AD mice, age 3 months, without treatment (3×Tg-3m); (3) Wild type mice, age 3 months, treated with vehicle feed without P021 until the age of 21 months (WT-21m/Veh); (4) 3×Tg-AD-mice, age 3 months, treated with vehicle feed without P021 until the age of 21 months (3×Tg-21m/Veh); and (5) 3×Tg-AD-mice, age 3 months, treated with 60 nmol P021/g feed till the age of 21 months (3×Tg-21m/P021). The first two groups were the starting point and were employed as the matched young controls. P021 was formulated in the feed by Research Diets (New Brunswick, N.J., USA). Food consumption and body weight were recorded every 2 weeks and every month, respectively. The average mouse food consumption was ˜2.7 g feed/day.

Tissue Processing

The animals were anesthetized by using an overdose of avertin and then were transcardially perfused by using 0.1 M phosphate-buffered saline (PBS). Eyeballs were dissected out from the carcasses, immersion-fixed for 24 hours in 4% paraformaldehyde at 4° C., and then transferred to 70% alcohol for storage; in the case of mice, the eyeballs were prefixed by injecting 4% paraformaldehyde, followed by removal from the carcasses and immersion fixation as above. After the cornea and lens of the left eyes were removed, the remaining eye cup was embedded in paraffin with the orientation parallel to the optic nerve in longitudinal position. In all cases, 5-μm serial sections were cut by using a rotary microtome and were mounted on Superfrost Plus slides.

Histological Analysis

Every 25^{th} retinal section was deparaffinized and subjected to hematoxylin and eosin (H & E) staining to evaluate the morphology. The H & E staining images were captured with a light microscope using 2×, 40×, and 100× objective lenses. Because there is no significant difference between the ⅕th to the ⅓rd of the retina from the optic nerve head and the most peripheral part, the ⅕^{th} position of the retina from the optic nerve to the most peripheral part of the retina was chosen as the “central,” and the most peripheral part of the retina as “peripheral.” Rows of the outer nuclear layer (ONL) and the inner nuclear layer (INL) of the retina in each group (every 25^{th} section, 3-5 sections per rat or mouse) were manually counted in three columns per image by using a 40× or, where necessary, a higher magnification objective. The thicknesses of the ONL and INL of the central and peripheral retina in each group (every 25th section from the optic nerve head, 2-3 sections per rat or mouse) were measured and quantified by using the ImageJ/NIH image analysis system.

Autofluorescence

Paraffin sections were deparaffinized, rehydrated, and covered with coverslips with 0.1M PBS and then photographed by using a confocal microscope with red channel (λₑₓ=543, λₑₘ=590/50), as described previously. To analyze the lipofuscin granules in RPE and the deposits between RPE and choroid (Cho) (142-2002, Marmorstein et al., 2002), the sections were photographed by using a 60× objective lens. For quantification, the number of deposits between RPE and Cho in each section was manually counted by using a 20× or, where necessary, a higher magnification objective.

Immunofluorescence

Paraffin sections were deparaffinized, rehydrated, and subjected to antigen retrieval by boiling for 20 minutes in 10 mM sodium citrate solution (pH 6.0). Sections were then washed three times in 10 mM PBS for 15 minutes each and incubated in 0.3% Triton X-100 for 30 minutes. The sections were again washed in 10 mM PBS (15 minutes, three times each) and blocked in blocking solution (5% normal goat serum containing 0.1% Triton X-100 and 0.05% Tween 20 in PBS) for 45 minutes. Sections were then incubated overnight at 4° C. with the corresponding primary antibodies (see Table 1 for antibodies used in this study) diluted in the same blocking solution. After being washed three times for 15 minutes each with 10 mM PBS, sections were incubated with Alexa-Fluor 488-conjugated goat anti-mouse or anti-rabbit IgG secondary antibodies (1:1000) in 10 mM PBS with 0.05% Tween 20 for 2 hours at room temperature. Sections were subsequently washed one time and incubated with To-Pro (1:1000) for 15 minutes. After being washed three times for 15 minutes, sections were mounted and cover-slipped by using ProLong Gold anti-fade reagent. The absence of primary antibody control staining was included in each experiment as a negative control. Double immunofluorescence and To-Pro images were captured at equal camera exposure (for each antibody staining) with a Nikon EZ-C1 laser confocal imaging system. The area to be analyzed was selected and converted to grey scale, the threshold (Yen mode) was adjusted, and the area percentage of positive staining was measured for each section by using the NIH Image J software package. The staining area was averaged from 2-3 independent sections of each eye (n=5-7 different animals per group).

Statistical Analysis

Data were analyzed by using Prism version 5.0 software (Graph Pad Software Inc., La Jolla, Calif., USA) and one-way ANOVA followed by Newman-Keuls Multiple Comparison Tests. All data were computed as mean±SEM. P<0.05 was considered statistically significant.

Results

P021 Rescues the Retinal Pathology in Aged Fisher Rats

At as early as 12 months of age, OXYS rats and Wistar rats are known to show AMD-like pathology, such as BM thickening and lipofuscin accumulation. Previous evaluations of the effect of chronic oral treatment with P021 on tau pathology and cognitive impairment in aged rats and mice did not reveal any worsening in general physical state because of P021 treatment, suggesting a probable lack of any side effects. Here, to assess the AMD-like retinal pathology induced by aging and to study the effect of P021 on the pathology, AMD features in rats were investigated. Young (˜2-3 months of age) and aged (˜19-21 m) female Fisher rats were chosen for this study. Briefly, the aged rats were administered P021 (500 nmoles/kg/day) in saline or vehicle only by gavage, and the young rats were treated with vehicle only for 3 months (FIG. 1A). The morphological changes in sections of central and peripheral retinas were studied after 3 months' treatment (FIG. 1B, C). Photoreceptor cell loss was evaluated by analyzing the rows and thickness of the layers of photoreceptor cell nuclei (ONL). H & E staining showed that rows and thickness of ONL in the central and peripheral retinas were dramatically decreased in 22- to 24-month-old rats (FIG. 1C, D, F). P021 prevented these pathological changes more effectively in the central retina (FIG. 1D, F) than in the peripheral retina (FIG. 3D, F). There was also no significant difference between ˜5- to 6-month-old rats and P021-treated ˜22- to 24-month-old rats (FIG. 1D, F), which suggests that P021 can prevent the ONL lesions. Also, the rows and thickness of INL in the peripheral retina were decreased in the ˜22- to 24-month-old/Veh rats compared with the ˜5- to 6-month-old/Veh rats, and ˜22- to 24-month-old/P021 rats showed a clear trend to rescue the number of rows (FIGS. 1 C and E, G). There were no significant differences in the rows and thickness of INL in the central retina between ˜5- to 6-month-old/Veh and ˜22- to 24-month-old/Veh rats, and P021 had no detectable effect (FIGS. 1C and E, G). Additionally, the laminar arrangement in the central retina in ˜22- to 24-month-old/Veh rats was distorted by outer retinal folds and rosette-like structures (FIG. 1 C-b). These aberrations involved a semi-spherical organization of photoreceptor cell nuclei that occupied the ONL, extended across the outer plexiform layer (OPL), and encroached the INL. The center of the rosettes appeared to be occupied by photoreceptor inner segment (IS). No abnormal structure like this was observed in the ˜5- to 6-month-old/Veh rats or ˜22- to 24-month-old/P021 rats (FIG. 1C-a, c, d, f). Altogether, these results indicated photoreceptor cell degeneration in the central retina, and INL degeneration in the peripheral retina in ˜22- to 24-month-old/Veh rats and suggested that 3-month-long treatment with P021 protected ˜19- to 21-month-old rats against AMD-like pathology in the central retina.

P021 Rescues the Pathophysiology of RPE and BM in Aged Rats

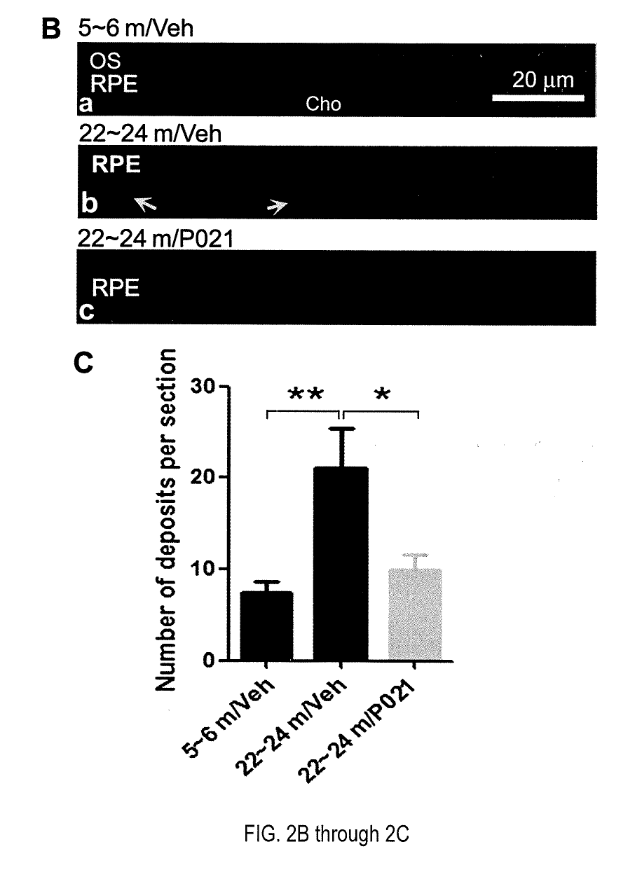

Dysfunction of the RPE presages photoreceptor cell loss, and the histological abnormalities in RPE are also a hallmark of human dry AMD. To investigate the histopathological changes in rats, the RPE layer was examined in sagittal sections stained with H & E. Apical microvilli (AM) in RPE were apparent in ˜5- to 6-month-old/Veh rats (FIG. 2 A-a); in contrast, they were shortened or disorganized in the ˜22- to 24-month-old/Veh rats (FIGS. 2 A-b-d), and P021 treatment showed improvement in the arrangement of AM. Additionally, many RPE cells were vacuolated (FIG. 2 A-c, yellow arrows), and RPE atrophy (FIG. 2 A-c, yellow RPE) was detected in aged rats, whereas no apparent abnormalities were observed in the RPE of ˜5- to 6-month-old/Veh rats and ˜22- to 24-month-old/P021 rats (FIG. 2 A-a, d). Furthermore, many lipofuscin granules (FIG. 2A-c; FIG. 2B-b) and deposits (FIGS. 2B-b and C) were present between RPE and Cho detected by auto-fluorescence in ˜22- to 24-month-old/Veh rats, whereas no lipofuscin granules were detected in ˜5- to 6-month-old/Veh rats (FIG. 2A, and FIG. 2 B-a); lipofuscin granules and deposition number were obviously decreased in ˜22- to 24-month-old/P021 rats (FIGS. 2 B-c, and C). These results suggest that ˜22- to 24-month-old/Veh rats exhibit RPE abnormalities, and P021 ameliorates these pathological changes.

Chronic Treatment with P021 can Prevent Retinal Lesions in Aged Mice

AMD-like pathology was reported previously in Tg2576-mice and 5×FAD mice. The occurrence of AMD-like changes in 3×Tg-AD mice (3×Tg-AD) was not known. Here, the presence of AMD-like pathology and its prevention by P021 treatment in aged 3×Tg-AD mice was investigated.

To evaluate the effect of P021 on the development of retinal pathology, 3-month-old female 3×Tg-AD mice were fed with formulated feed containing P021 (60 nmoles/g feed) or vehicle feed until they reached 21 months of age. WT-3 m and 3×Tg-3 m mice were used without any treatment as young controls (FIG. 3A). Paraffin sections of the eyecup parallel to the axis of the pupil to the optic nerve were employed to study changes in the retina (FIG. 3 Ba-e). The rows and thickness of ONL were all significantly decreased, both in the central retina and in the peripheral retina in WT-21 m/Veh and 3×Tg-21 m/Veh mice and that these changes in the central retina, and not in the peripheral retina, were prevented in 3×Tg-21 m/P021 mice (FIGS. 3 3B, 3C, 3E). Rows and thickness of INL in the central retina were also decreased in WT-21 m/Veh and 3×Tg-21 m/Veh mice, and chronic treatment with P021 prevented these changes in 3×Tg-21 m mice; although there was an obvious trend towards statistical significance, the rescue of INL thickness by P021 treatment did not reach statistical significance (FIG. 3B, 3D, 3F). The age-associated changes in rows and thickness of INL in the peripheral retina were smaller than those seen in the central retina, and their prevention by chronic treatment with P021 did not reach significance in 3×Tg-21 m/P021 mice (FIG. 3B, 3D,3 F). In addition, the nuclei of ONL in the central retina were extended across the OPL or protruded into the INL in the WT-21 m/Veh and 3×Tg-21 m/Veh mice, which made the gap between the ONL and the INL narrower or unclear (FIG. 3Bh, i), whereas this pathology was prevented in 3×Tg-21 m/P021 mice (FIG. 3Bj). Altogether, these findings indicated the degeneration of both the ONL and the INL in the central and peripheral retinas in WT/Veh and 3×Tg/Veh mice at 21 months of age, and the prevention of these changes, especially in the central retina, by chronic treatment with P021 in 3×Tg-AD mice.

P021 Prevents RPE and BM Pathology in Aged Mice

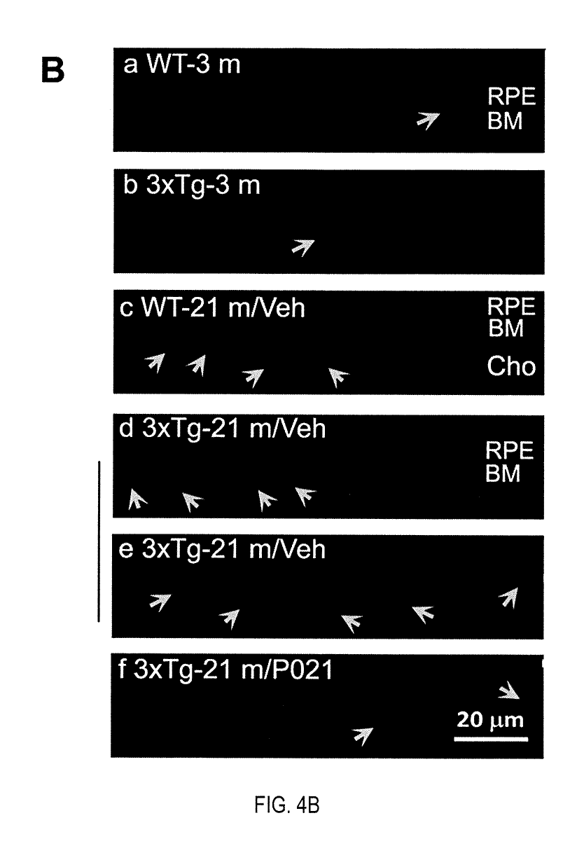

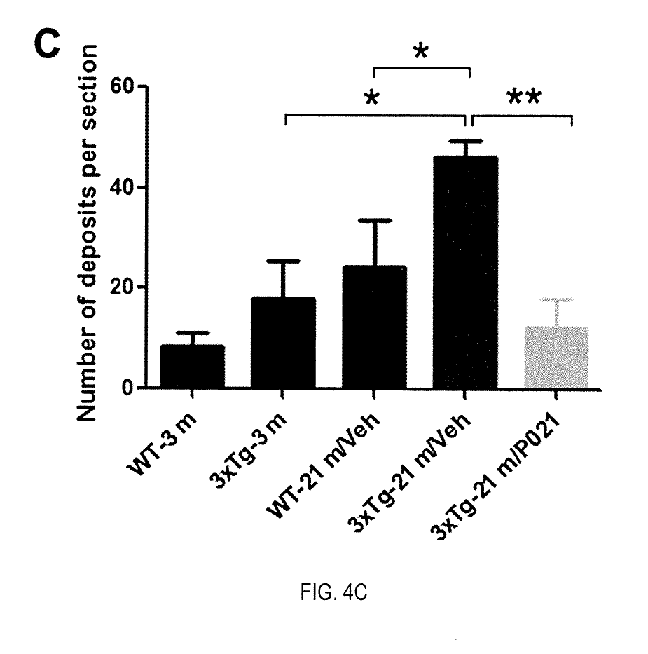

Disruption and degeneration of RPE is well known to occur in AMD. The occurrence of this pathology was investigated in both aged WT and 3×Tg-AD mice and the effect of chronic treatment with P021 on its prevention. The areas with the greatest photoreceptor disorganization were often associated with atrophied RPE and increase in lipofuscin granules in the central retina in 3×Tg-21 m/Veh mice (FIG. 3 B, i). The central retina in WT-21 m/Veh and 3×Tg-21 m/Veh mice showed multiple additional AMD features, including hypo-pigmentation, thinning, and disorganization of RPE (FIG. 3B, i, FIG. 4A, d-e). RPE in 3×Tg-21 m/Veh mice also showed frequent accumulation of large lipofuscin granules (FIG. 3B-i and FIG. 4A-e). An increase in auto-fluorescence attributable to lipofuscin granules was found in the aged mice (FIG. 4 B-c, d, e). Thickening of the BM was also found in 3×Tg-21 m/Veh mice (FIG. 4A-d). Deposits between the RPE and Cho were detected by auto-fluorescence in 3×Tg-21 m/Veh mice, much more than in WT-21 m/Veh mice (FIG. 4 B-d, e); WT-3 m or 3×Tg-3 m mice controls, however, showed very few deposits (FIG. 4B-a, b). The deposits were significantly reduced in 3×Tg-21 m/P021 mice, although some small lipofuscin granules could still be observed (FIG. 4 B-f). Together, our data indicate that aged mice, especially 3×Tg-21 m/Veh mice, exhibit RPE and BM abnormalities that highly resemble human dry AMD. Most of these AMD features were prevented by chronic treatment with P021 administered in the diet.

P021 Reduces the Microgliosis in the Retina of Aged Mice and Rats

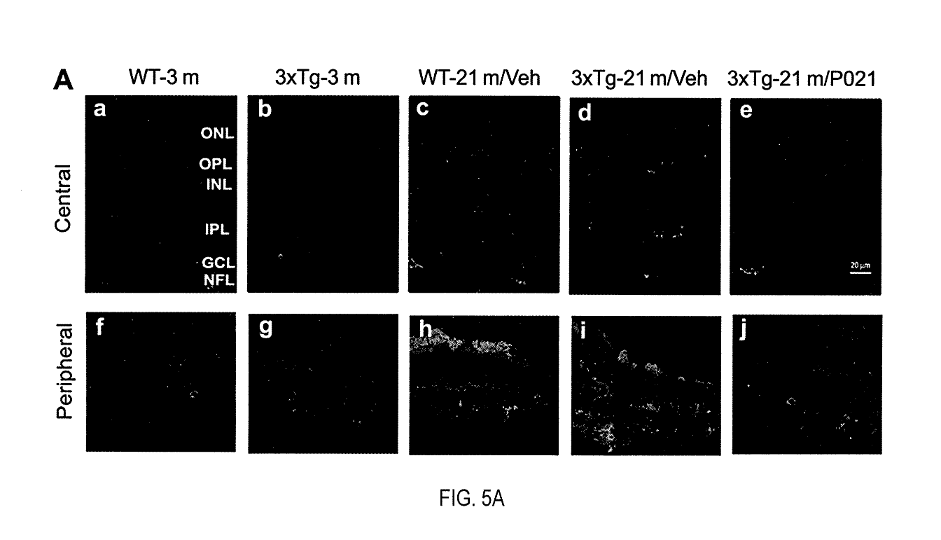

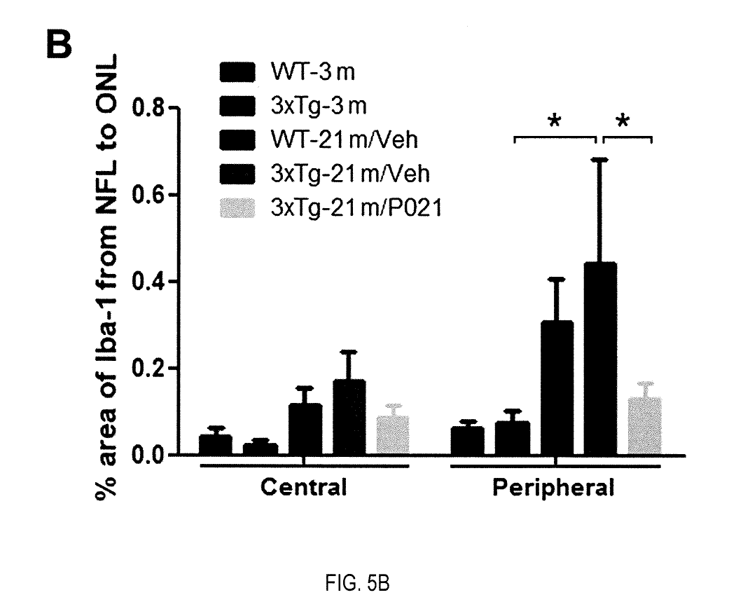

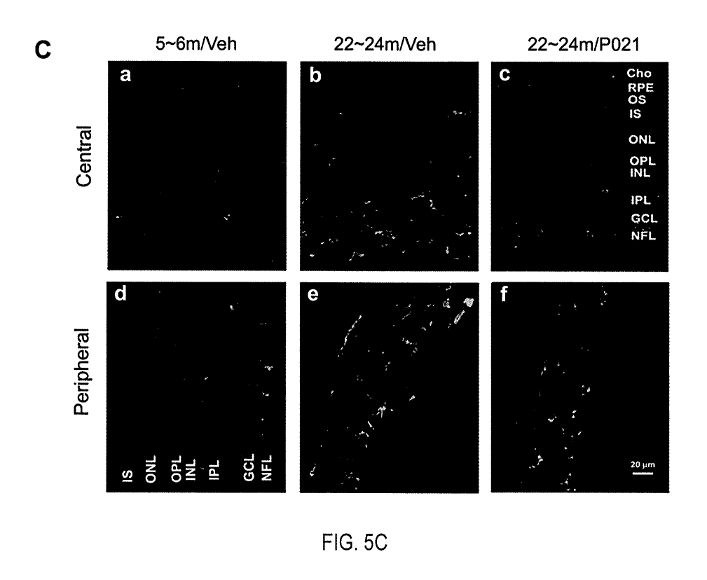

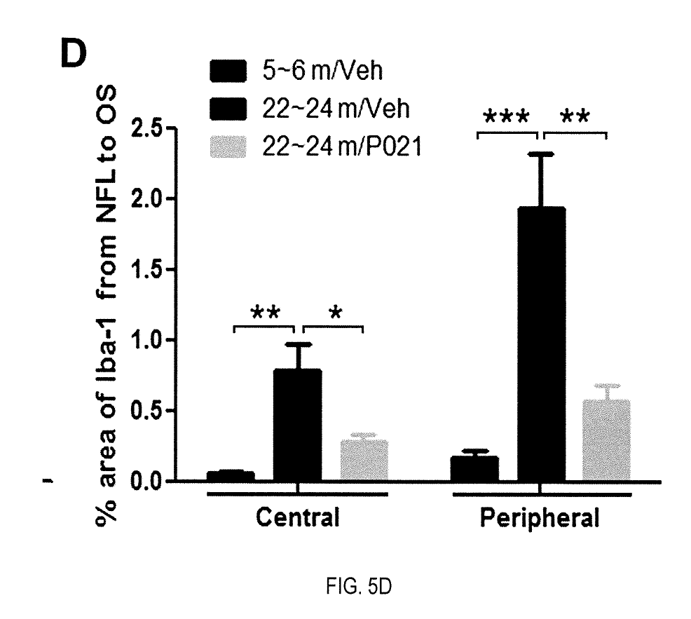

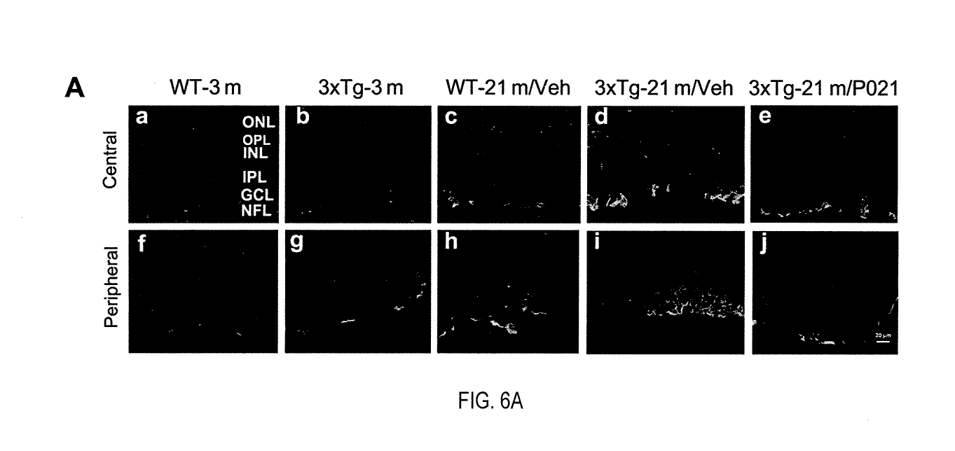

Microglial cells form an important part of the immune defense of the retina. In the normal adult retina, microglia are quiescent, composed of small and stellate cells strictly limited to the INLs. In AMD, activated microglia are found in the ONLs, associated with photoreceptor degeneration, suggesting a role in the removal of cell debris. Iba-1 immunofluorescence was used to study microgliosis in the retina of aged mice and rats. Iba-1-positive cells were located mainly in the inner layers of the retina (FIG. 5). In WT-21 m/Veh mice and 3×Tg-21 m/Veh mice, Iba-1-positive staining was distributed into the nerve fiber layer (NFL), ganglion cell layer (GCL), inner plexiform layer (IPL), OPL, and even into the ONL of the central retina (FIG. 5 A-c, d), whereas in WT-3 m, 3×Tg-3 m, and 3×Tg-21 m/P021 mice, Iba-1-positive staining was only observed in the GCL; very few Iba-1-positive cells were found in the IPL and other layers (FIG. 5A-a, b, e). Moreover, there was an obvious trend of increase in Iba-1 immunofluorescence from the NFL to the ONL in the central retina in WT-21 m/Veh and 3×Tg-21 m/Veh mice (FIG. 5B). Although the area percentage of Iba-1-positive staining in the peripheral retina of WT-21 m/Veh, and 3×Tg-21 m/Veh was higher than in the young controls (WT-3 m and 3×Tg-3 m), these changes showed a trend towards statistical significance, but were not statistically significant. Interestingly, the Iba-1 immunostaining in 3×Tg-21 m/Veh mice was significantly higher than in WT-21 m/Veh mice, whereas it was decreased in 3×Tg-21 m/P021 mice to the levels in WT-3 m/Veh and 3×Tg-3 m/Veh mice in the peripheral retina (FIG. 5B), even though there was no obvious difference in the distribution of Iba-1-positive staining (FIG. 5A-f-j).

Iba-1-positive microglial cells were found not only in the whole retinal layers, including the NFL, IPL, INL, OPL, ONL, and even the IS/outer segment (OS), but also in the Cho of the ˜22- to 24-month-old/Veh rats (FIG. 5C-b, e), but the 5-6 m/Veh rats showed a limited location of Iba-1 only from the NFL to the INL, and the Cho (FIG. 5C-a, d). In contrast, 22- to 24-month-old/P021 rats showed a decrease in localization from the NFL to the OPL compared with ˜22- to 24-month-old/Veh rats, and no staining was found from the ONL to the OS, especially in the central retina after P021 treatment (FIG. 5C, c, f). Quantitatively, the relative Iba-1 immunostaining from the NFL to the OS in the central and peripheral retinas of 22- to 24-month-old/Veh rats was increased very much in ˜22- to 24-month-old/Veh rats as compared to ˜5- to 6-month-old/Veh rats, and P021 treatment dramatically reduced it in ˜22- to 24-month-old rats (FIG. 5D)

Altogether, these findings indicate that microglia are distributed more widely in the whole retina of aged mice and rats, especially in the ONL, which is associated with photoreceptor degeneration (shown in FIGS. 1 and 3), and that treatment with P021 can efficiently protect the retina from the inflammatory damage in aged mice and rats.

3.6. P021 Reduces Astrogliosis in the Retina of Aged Mice and Rats

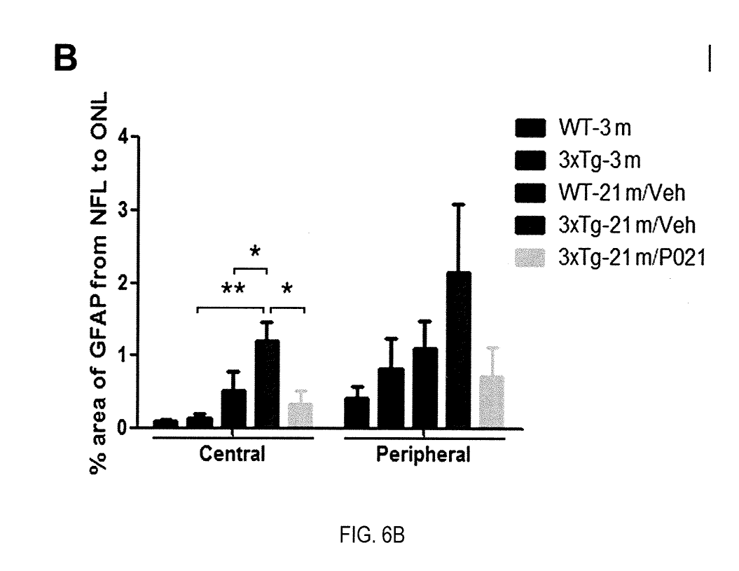

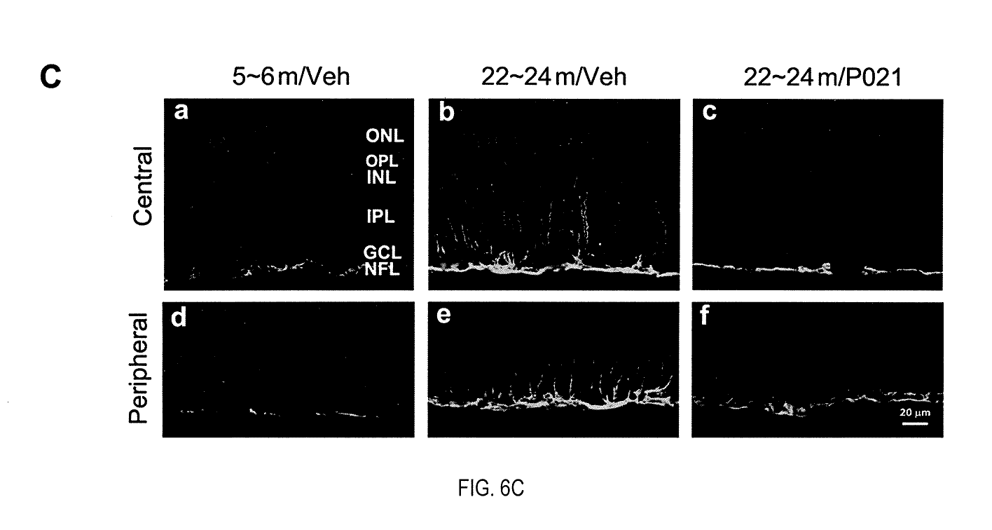

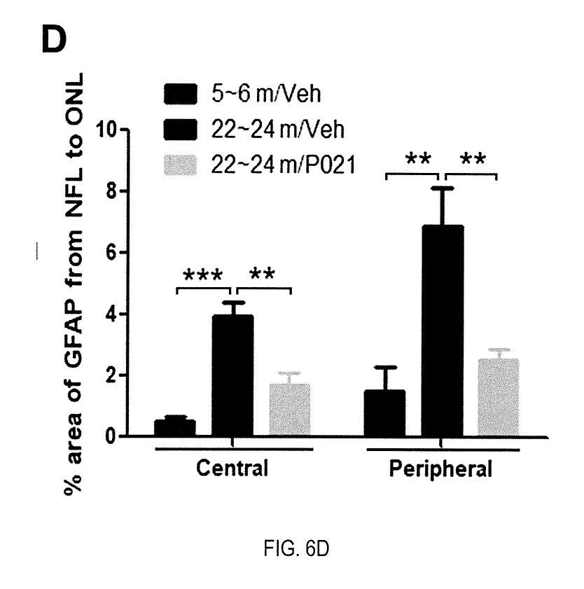

Astrocytes are another key cell type involved in macular degeneration. Previous studies showed activated astrocytes in the retina of Tg2576 mice at 14 months and of 3×Tg-AD mice at 9 months and ˜18-24 months. To study the localization of astrogliosis and the response to P021 treatment in the retina of aged mice and rats, astrocytic/Müller cell activation was evaluated by immunostaining with anti-glial fibrillary acidic protein (GFAP). GFAP-positive staining was confined to astrocytes in the GCL of young mice (FIG. 6A-a-b, f-g) but was highly elevated in Müller glial cells in the INL and other layers such as the IPL, OPL, and ONL of the central and peripheral retina in 3×Tg-21 m/Veh mice (FIG. 6A-d,i). In contrast, the GFAP-positive staining was limited to the NFL and GCL in 3×Tg-21 m/P021 mice (FIG. 6A-e, g). The area percentage of GFAP from the NFL to the ONL in the central retina of 3×Tg-21 m/Veh mice was significantly higher than in 3×Tg-3 m mice and WT-21 m/Veh mice, and was reduced by P021 treatment to the levels in WT-3m/Veh and 3×Tg-3 m/Veh mice (FIG. 6B). There was also a similar trend in the peripheral retina between groups, but the changes did not reach statistical significance (FIG. 6B).

Retinal astrogliosis was studied by GFAP immunostaining in rats. The area of GFAP-positive staining was extended from the NFL to the ONL in the central and peripheral retinas of ˜22- to 24-month-old/Veh rats (FIG. 6 C-b, e), but the staining was considerably less and was limited to the NFL and GCL in ˜5- to 6-month-old/Veh rats (FIG. 6C-a, d). The GFAP staining was reduced very much in ˜22- to 24-month-old/P021 rats as compared to ˜22- to 24-month-old/Veh rats (FIG. 6C-c, f). The area percentage of GFAP-positive staining was also drastically elevated in the central and peripheral retinas in ˜22- to 24-month-old/Veh rats as compared to ˜5- to 6-month-old/Veh rats and ˜22- to 24-month-old/P021 rats (FIG. 6D).

Altogether, the above findings indicate that P021 can prevent and rescue microgliosis and astrogliosis in the retina of both aged 3×Tg-mice and aged rats.

P021 Affects Alzheimer-Like Pathology and VEGF Changes in the Retina and Optic Nerve of Aged Rats and Mice

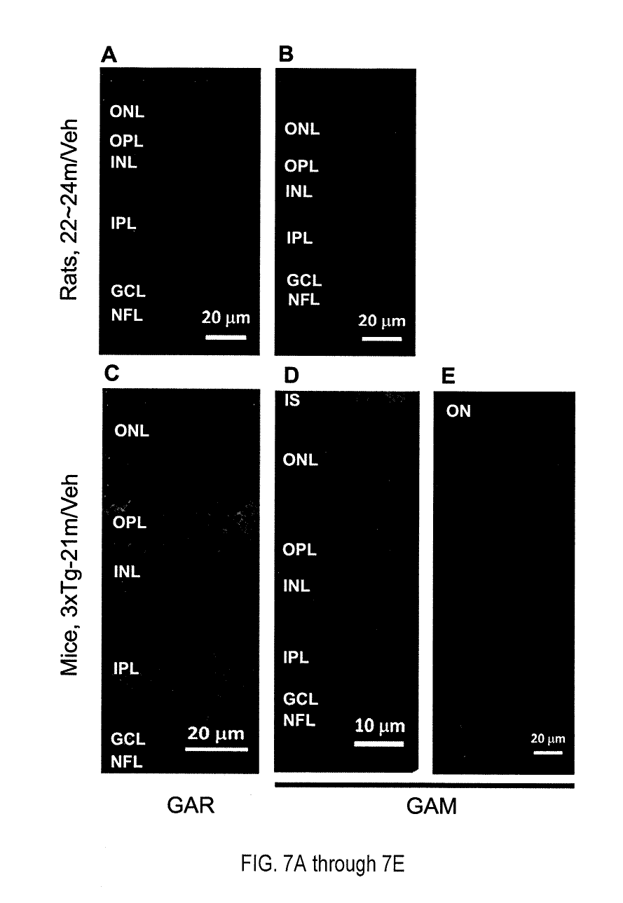

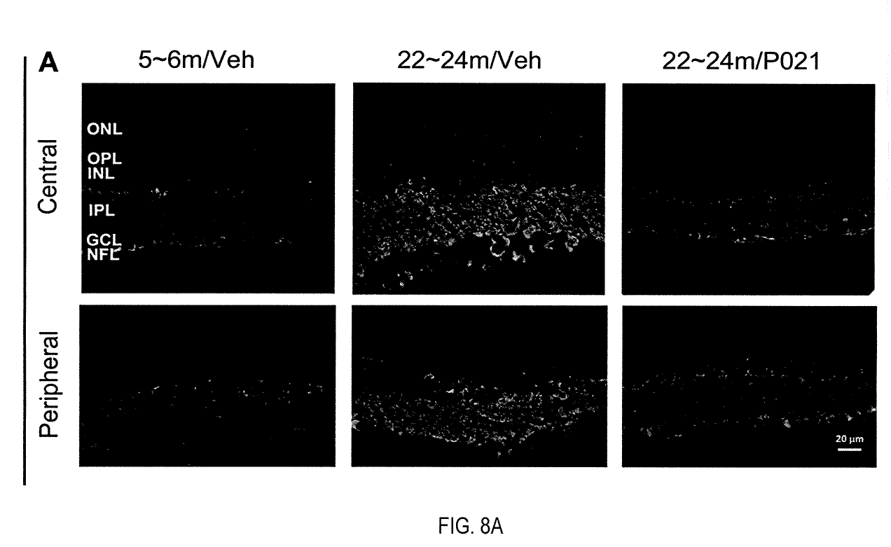

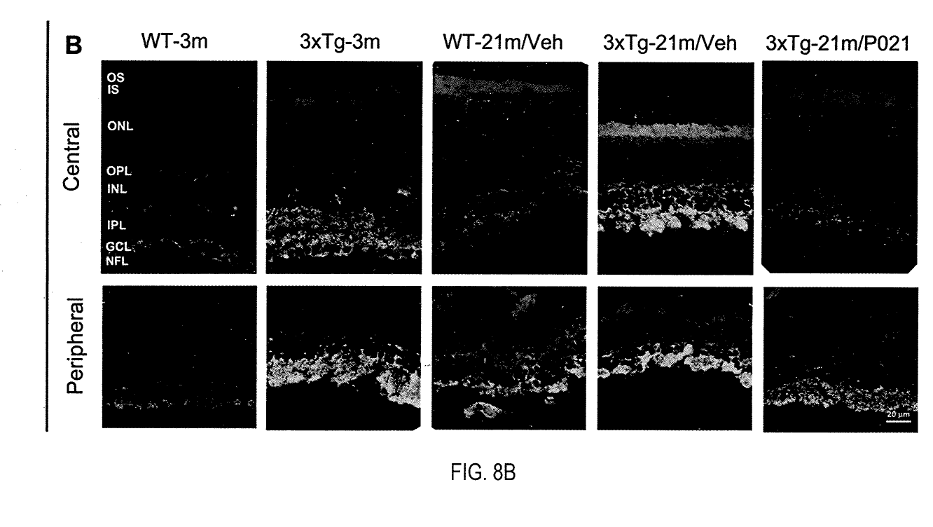

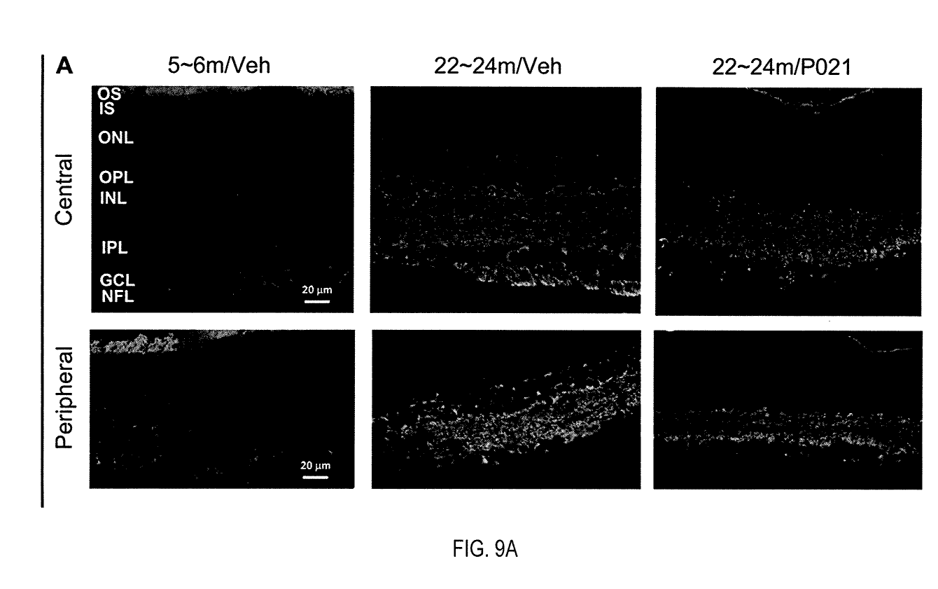

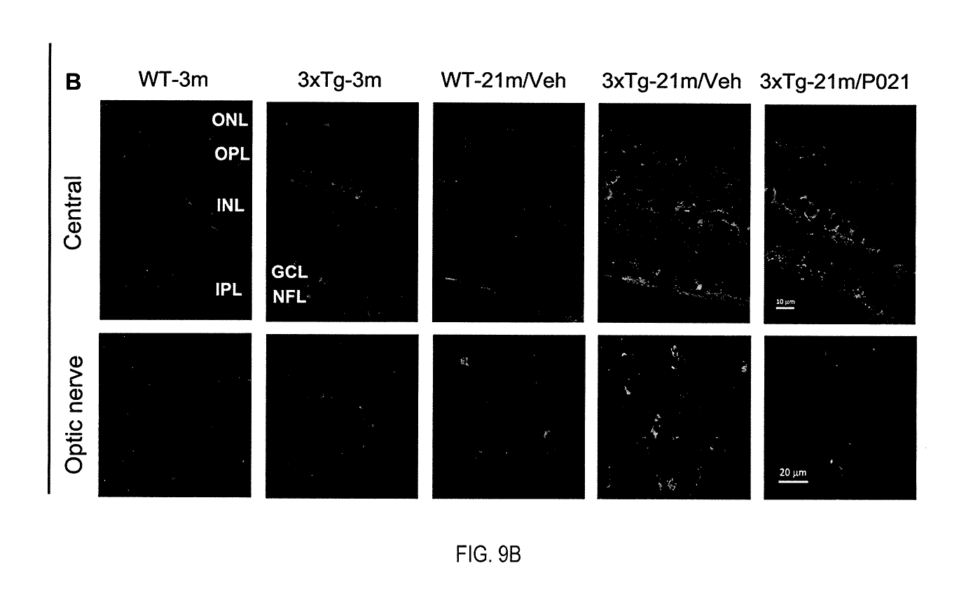

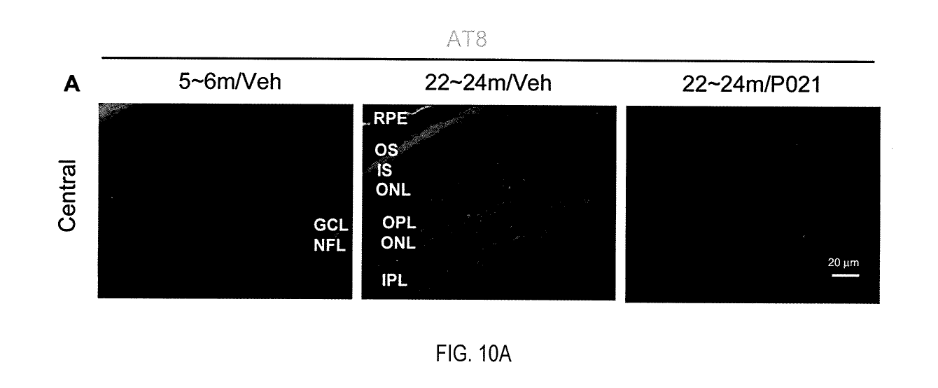

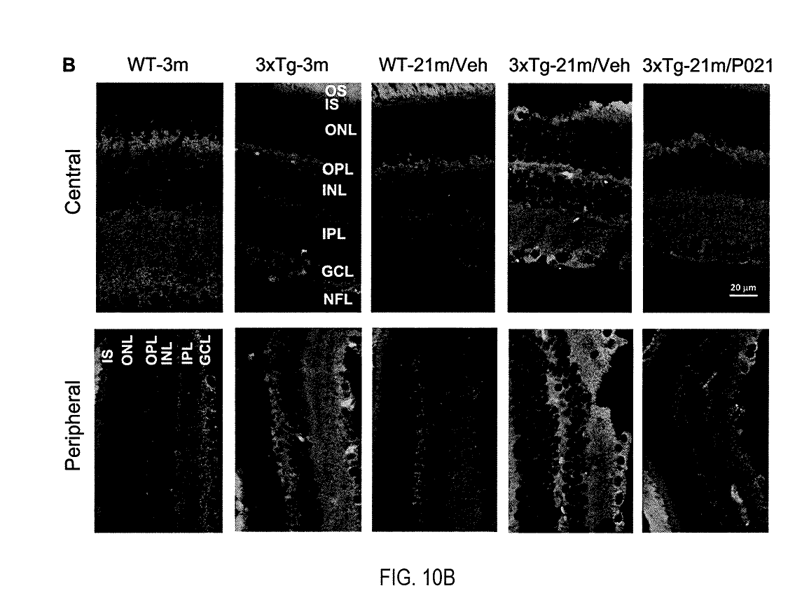

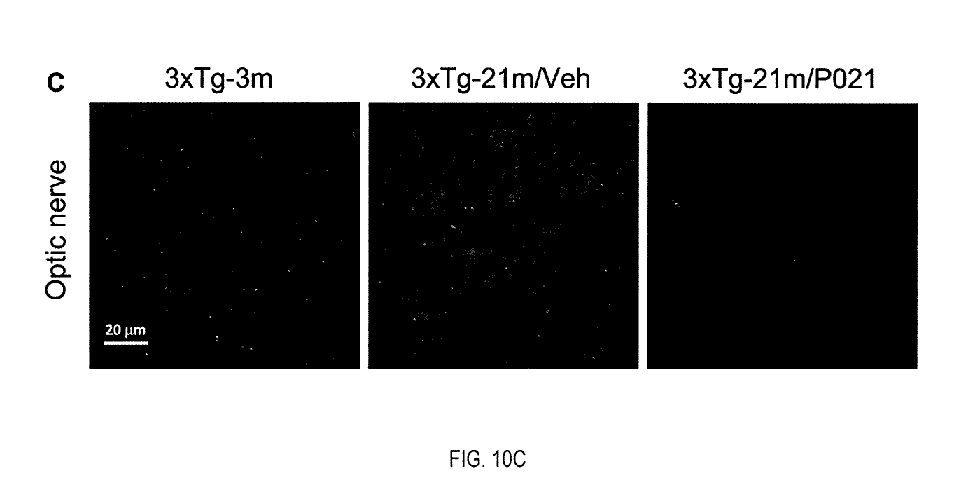

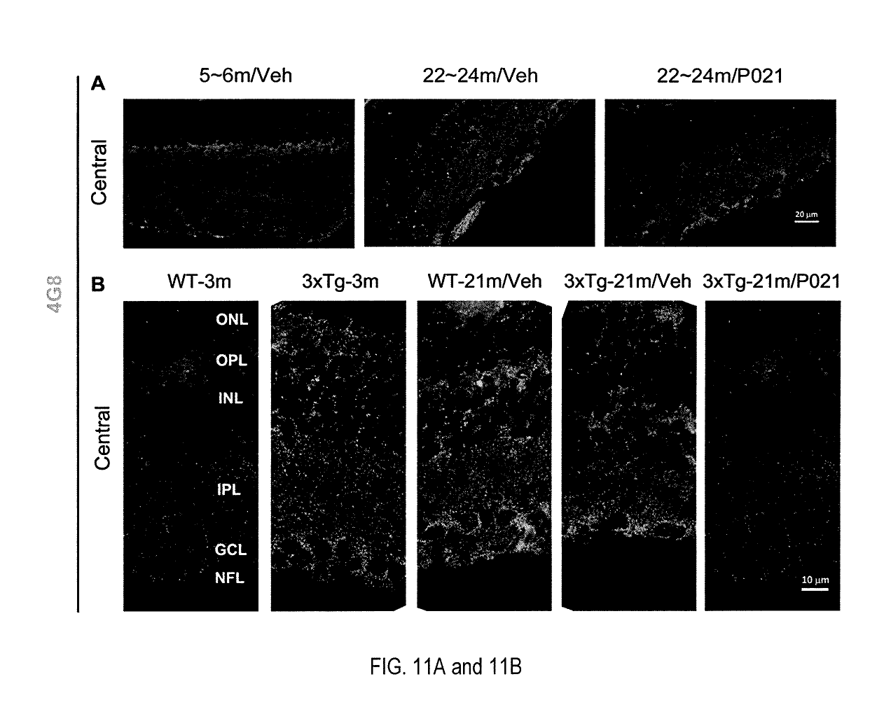

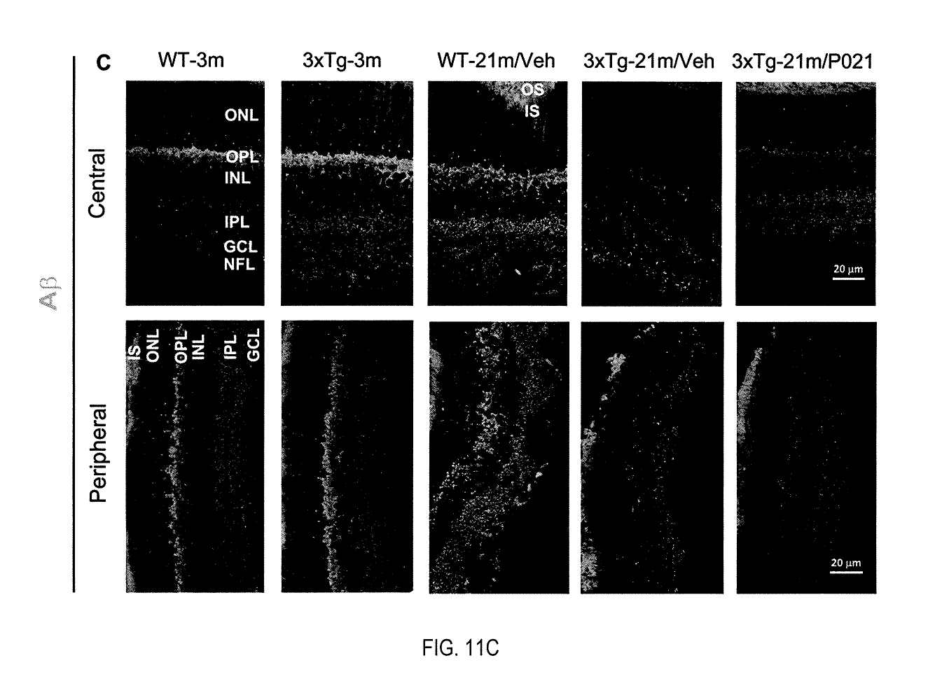

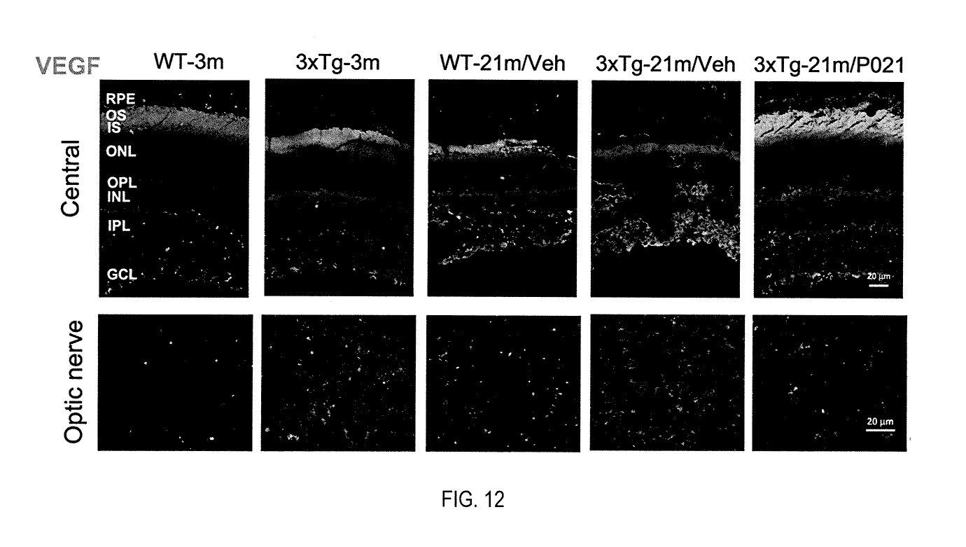

Tau and Aβ pathologies are well-known hallmarks of AD. Previous studies showed both tau and Aβ pathologies in the retina of 14m Tg2576 mice and AβPP immunoreactivity in the retina in a number of AD animal models, including Tg mouse models (hTgAPP^{tg/tg}, APP_{SWE}/PS1_{ΔE9}, and APP_{SWE}/PS1_{M146L/L286V}) that exhibit several features of AD. Moreover, several studies found Aβ deposition in the drusen or retinal layers in AMD animal models. Anti-Aβ immunotherapy was reported to reduce ocular Aβ deposits in a mouse model of AMD. In the present study, the presence and the effect of P021 treatment on AD pathology in the retina was investigated. Tau and Aβ pathologies in the retinas of aged rats and 3×Tg-mice were found: immunofluorescence results showed that after normalization with negative controls (FIG. 7), positive staining of total tau detected by rabbit polyclonal antibody R134d (FIG. 8) and hyperphosphorylation of tau at Ser-396/404 by mouse monoclonal antibody PHF-1 (FIG. 9) were increased in the sub-retinal layers of aged rats and 3×Tg-mice, and even 3×Tg-21 m/Veh showed increased PHF-1 immunoreactivity in the optic nerve (FIG. 9B, lower panel). Hyperphosphorylation of tau at Ser-202/Thr-205 detected with mouse monoclonal antibody AT8 (FIG. 10), and Aβ/APP detected by mouse monoclonal antibody 4G8 and rabbit monoclonal Aβ (FIG. 11) were also highly expressed in sub-retinal layers. The positive staining was not sharp and condensed, which appeared as small particles, especially PHF-1, AT8, 4G8, and Aβ immunostaining in the retina and optic nerve in mice, but compared with the negative controls (FIG. 7C, 7D), the positive staining was specific. Importantly, P021 treatment prevented the increase in tau and Aβ pathologies in the aged mice and rats.

Late-stage dry AMD and wet AMD can coexist in AMD development. Excessive amounts of vascular endothelial growth factor (VEGF) is a major pathological change in wet AMD, and anti-VEGF can relieve or delay the progress of wet AMD. Anti-VEGF treatment of the combined dry/wet AMD phenotype was reported to be effective in a case series of 11 eyes. In the present study, VEGF-positive spots were found to be located mainly in the inner retinal layers from the NFL to the IPL and in the RPE in young mice, but they were more widely distributed in the INL, OPL, and ONL in aged mice and less so in the RPE in aged WT-21 m/Veh and 3×Tg-21 m/Veh mice. In 3×Tg-21 m/P021 mice, the localization of VEGF-positive spots was limited mainly to the GFL to the IPL, similar to in the young control animals. In the optic nerve, aged 3×Tg-21m/Veh mice showed an increase in VEGF staining, and the size of the staining patch was decreased after P021 treatment.

Discussion

In this study, several AMD-like features that recapitulate human dry AMD were found for the first time, including photoreceptor cell loss (decrease in rows and thickness of ONL), rosette-like formation in the photoreceptor cell layer, RPE disruption, accumulation of lipofuscin and vacuoles in RPE, increase in auto-fluorescence of RPE, BM thickening, and the formation of basal deposits between RPE/BM and Cho in aged rats and 3×Tg-AD mice. P021 conferred protection for the retina against this age- and disease-related damage. Even neuroinflammation detected by microgliosis and astrocytosis was ameliorated by P021 treatment. Furthermore, P021 prevented the increase in tau and Aβ pathologies and VEGF deposition in the sub-retina.

These findings are the first demonstration of AMD-like pathology in aged rats and 3×Tg-AD mice and its rescue by a neuro-regenerative compound, P021.

Several studies reported a decrease in rows and thickness of the ONL or retinal thickness in different animal models of AMD. The thickness of the ONL in 24-month-old mice treated with a high glycemic diet was decreased. Rows of ONL were reduced in 4-month-old C57BL/6 mice after intraocular injection of Aβ40/42. Another group found that retinal thickness was significantly decreased in 14-month-old Tg2576 mice. Our finding of the decreased number of rows and reduced thickness in the ONL seen by H & E staining in aged rats and mice is in agreement with these findings. Moreover, consistent with these findings, the present study also showed, by Iba-1 and GFAP immunofluorescence counterstained with To-Pro, a decrease in the rows and thickness of the ONL and INL in aged mice and rats, and P021 treatment prevented these changes. These degenerative changes in the ONL and INL might occur only in the aged animals, because these changes were not found in 3-month-old mice or in 5- to 6-month-old rats, and a previous study reported no significant changes in rows and thickness of the retinal layer in APP_{SWE}/PS1_{ΔE9} mice at ˜9-12 months of age.

RPE pathology is one of the pivotal AMD features, and drusen or drusen-like deposition between RPE and BM was reported to in AMD in humans and some animal models. In the present study, drusen-like pathology was not found in aged 3×Tg-mice and in aged rats, which is possibly because of the small sample size or the animal models used. Actually, not all models of AMD exhibit the typical morphology, and drusen is not uniquely associated with AMD. Interestingly, a rosette-like formation was found in ˜22- to 24-month-old rats, and it was similar to the previous findings that were reported in the retina of a 92-year-old male with AMD and in a 3-month-old Rdh8^{-/-}/Abca4^{-/-} mouse retina stained with H & E. The rosette-like rearrangements of photoreceptor cells extend from the ONL and across the OPL toward the INL, and photoreceptor inner segments or partial outer segments project inward to form the core of the rosette. Our finding of rosette-like structures is consistent with the disorganized and fragmented ONL or photoreceptors reported in 24-month-old OXYS rats.

In addition, BM thickening, atrophy, and vacuolization of RPE was observed in 21-month-old 3×Tg/Veh-mice and ˜18- to 24-month-old rats, which is consistent with previous studies in 17-month-old Cxcr5^{-/-} mice and a 27-month-old neprilysin-deficient mouse model of AMD. Senescence-accelerated OXYS rats had atrophic areas in the RPE at 1.5 months of age, and some animals developed thickened BM by 12 months, and obvious atrophy at 24 months. However, it is important to note that RPE vacuolization in human AMD is actually quite rare and is not considered a phenotypic feature of the disease, although it has been reported in mouse models of AMD.

In spite of some studies that reported that lipofuscin deposits develop with aging, plenty of reports also showed that lipofuscin accumulation in the RPE is an important change in individuals with AMD and in mouse models. Increased lipofuscin granules in RPE cells were also observed in 11- to 13-month-old OXYS rats and in 24-month-old Wistar rats. In the present study, it was found that not only H & E staining but also auto-fluorescence exhibited a drastic increase in lipofuscin particles in the RPE and a large amount of deposits between the RPE/BM and Cho in aged rats and 3×Tg-mice. These findings suggest that preferential accumulation of lipofuscin in the RPE or the deposits outside the RPE may be due to, to a limited extent, the increased phagocytic and metabolic load on the RPE, which ultimately leads to photoreceptor cell loss or degeneration of other cellular layers, as was reported previously.

Clinically, fundus auto-fluorescence has proven to be valuable in the diagnosis and differentiation of retinal disease, and post-mortem AMD retinas showed increased auto-fluorescence in the RPE, BM, or Cho structures more efficiently when excited at 510 nm than at 470 nm. In the present study, the formation of thickened BM detected by H & E and auto-fluorescence was detected at λₑₓ=542 nm, and the increased auto-fluorescence in the OS and IS was found in aged mice. The RPE lipofuscin is known to be produced in the membranes of outer segments from the non-enzymatic reactions of vitamin A aldehyde. This fluorescent material is transferred to RPE cells within phagocytosed OS disks and becomes deposited in the lysosomal compartment of the cells or secreted to the outside of the RPE. These granules are known to have a strong phototoxic potential mediated by light-dependent reactive oxygen species (ROS) generation, and they could be a reason for further RPE degeneration.

Neuroglial cells are fundamental for the pathological progression of AD through multiple reactions, including astrogliosis, and microglial activation. In contrast, in the normal adult retina, microglia are quiescent, composed of small and stellate cells strictly limited to the inner retinal layers. The present study showed that in the retinas of WT-21m and 3×Tg-21m mice and ˜18- to 24-month-old rats, the distribution and intensity of microglial cells in the sub-retinal layers were higher than those in the young controls. The increased micorogliosis and wide distribution in aged mice and rats are partially in agreement with findings in the previous studies in different AMD models.

Impairment of microglial migration into or out of the sub-retinal space is known to promote the death of photoreceptor cells. In AMD, microglia accumulate in the subretinal space; this is probably a symptom of inflammatory damage, and the impairment of the accumulation in the sub-retinal space exacerbates retinal degeneration.

Another key glia cell type in the retina is astrocytes, and it was found that distribution and intensity of GFAP were significantly elevated in the whole retinal layers and displayed a hypertrophic and reactive morphology in aged WT and 3×Tg-mice and rats. These findings support the earlier report of increased GFAP-positive cell processes in the retinas of 3×Tg-AD mice ˜18-24 months of age compared to those seen at 9 months. The data presented here suggest that Müller cells and astrocytes in the AD retina undergo complex remodeling similar to astrocyte changes in the AD brain. Further studies are required to fully understand the consequences of glial activation in 3×Tg-AD mouse retinas and to determine how these changes correlate with tangles or Aβ amyloid deposition.

Numerous studies examining the retinas of sporadic and transgenic animal models of AD have reported Aβ deposits and hyperphosphorylated tau, often in association with retinal ganglion cell degeneration, local inflammation (i.e., microglial activation), and impairments of retinal structure and function, and increase in cytoplasmic AβPP in the photoreceptor layer in transgenic rodents. These studies, which included a variety of transgenic rat and mouse models, demonstrated abundant tau and Aβ deposits mainly in the innermost retinal layers (GCL and NFL). In the present study, the increase and wide distribution of total tau, phosphorylated tau and Aβ, and P021 treatment alleviated the pathology, which is consistent with our previous findings that P021 improves AD-like pathology in the brains of AD rat and mouse models. Although, in the current study, hyperphosphorylated tau and Aβ immunofluorescence was found in the retina of aged rats and 3×Tg-mice, both of them were weak and patchy; these findings were consistent with previous reports. In a previous study, tau was detected in the sub-retinal layers from the NFL to the OPL, even in the RPE of young and old humans, but the immunoreactivity was weak or patchy. Furthermore, it was suggested that Aβ and tau pathologies, combined with gliosis, drive neurodegeneration in AD.

In the present study, an increase in VEGF immunostaining was detected in the retinas of aged mice, which is consistent with previous studies that reported that anti-VEGF therapy is the standard of care for symptomatic wet AMD and can significantly improve visual acuity. Macrophages such as microglia, together with RPE cells, are a major source of pro-angiogenic factors, such as VEGF A, and the production of VEGF. In case of the breakdown of the blood-retina barrier, macrophage cells are recruited from the underlying Cho or from the systemic circulation into the retina, where they modulate disease. Thus, the increased microgliosis and astrogliosis found in the present study could have promoted the secretion and migration of VEGF into the subretinal layers and exacerbated the progression of dry AMD and led to the combined phenotype of dry/wet AMD.

Most of the pathological changes in morphology described above were prevented or reversed by P021 treatment, although some remained in the central retina and others in the peripheral retina. Also, microgliosis, astrogliosis, VEGF, and even tau/Aβ pathology in the sub-retina of aged rats and 3×Tg-AD mice were ameliorated by P021 treatment. Neuroinflammation in 3×Tg-AD mice has also been shown to correspond to ONL degeneration. Aβ or tau pathology may be the stimulators of neuroinflammation, according to previous reports related to AD. Moreover, the RPE is pivotal for maintaining the structure and function of the retina, and the early alteration of RPE cells may be a key factor for the development of the retinopathy in aged rats or mice and a cause of all subsequent pathological changes. Therefore, abnormalities of the retina might be initially caused by the enhanced neuroinflammatory response, including infiltration of inflammatory cells and local edema that leads to the RPE disruption and then the pathology in the sub-retinal layers. It is thus possible that P021, by virtue of its effect on retinal pathologies, normalized the tau/Aβ-induced neuroinflammation in aged 3×Tg-AD mice and rats, which in turn contributed to the beneficial effect of P021 on the pathological changes of the RPE, neuronal loss in the retina, especially in the ONL and INL, and even the whole retina.

The PI3K-Akt-GSK-3 pathway was reported to be insensitive in peripheral blood mononuclear cells of AMD patients and in cultured RPE cells, and in our previous study, P021 inhibited the tau and Aβ pathologies by increasing BDNF-mediated activation of TrkB-PI3K-Akt-GSK-3β signaling. Thus, in the present study, oral administration of P021 may have increased the activity of PI3K-Akt-GSK-3 signaling in the blood circulation system or in the RPE of the aged rats and mice, and then prevented the AMD-like pathology.

In the present study, aged rats and 3×Tg-AD mice were found to develop multiple pathological features of dry AMD from the aspect of morphology and immunohistochemistry. However, there are a few inherent limitations of this study. First, rodents do not have a macula/fovea and thus cannot completely mimic the human AMD condition. Second, no visual ability or electroretinography and biochemical analysis was conducted. Nevertheless, our study suggests that chronic treatment with P021 is an effective and attainable way to prevent or inhibit AMD-like pathology. Moreover, our findings demonstrate aged rats and 3×Tg-mice as new models of AMD that can be employed for preclinical studies to test compounds for therapeutic intervention of AMD.

What is claimed is:

1. A method of treating a subject for age related macular degeneration, comprising the step of administering a therapeutic amount of a peptidergic compound having the formula Ac-DGGL^{A}G-NH₂ (SEQ ID NO: 1).

2. The method of claim 1, wherein the therapeutic amount comprises a dosage of a dose of between 0.3 mg to 40 mg per kilogram of body weight.

3. The method of claim 2, further comprising the step of repeating the administering of the therapeutic amount of the compound on a daily basis.

4. The method of claim 3, wherein the step of repeating the administering of the therapeutic amount of the compound on a daily basis is performed for at least 90 days

5. The method of claim 4, wherein said compound is administered orally.

6. The method of claim 4, wherein said compound is administered intraocularly.

7. The method of claim 4, wherein said compound is administered intravenously.

8. The method of claim 4, wherein said compound is administered subcutaneously.
